(19) United States
(12) Patent Application Publication
(10) Pub. No.: US 2026/0098124 A1
(43) Pub. Date: Apr. 9, 2026

(54) Bio-Dihydroxyphenols for Use in Forming Bio-Liquid Crystalline Polymers

(71) Applicant: Ticona LLC, Florence, KY (US)

(72) Inventors: Xiaowei Zhang, Union, KY (US); Fangfang Tao, Shanghai (CN)

(21) Appl. No.: 19/112,618

(22) PCT Filed: Mar. 26, 2024
(86) PCT No.: PCT/CN2024/083726

§ 371 Date: Mar. 18, 2025

(51) Int. Cl. C08G 63/191; C07C 37/08; C07C 39/08

(52) U.S. Cl. CPC C08G 63/191; C07C 37/08; C07C 39/08

(57) ABSTRACT

A technique for forming a bio-based dihydroxyphenol monomer that is derived from bio-naphtha (e.g., “bio-BP”) and a bio-liquid crystalline polymer (“bio-LCP”) formed therefrom is provided.

### BACKGROUND OF THE INVENTION

Liquid crystalline polymers are commonly used in a wide variety of high performance applications, such as in high voltage connectors, medical products, camera modules, and so forth. Such polymers are typically produced by reacting one or more aromatic hydroxycarboxylic acids (e.g., 4-hydroxybenzoic acid (“HBA”) or 2-hydroxy-6-naphthoic acid (“HNA”)) and/or one or dicarboxylic acids (e.g., terephthalic acid (“TA”), isophthalic acid (“IA”), and 2,6-naphthylenedicarboxylic acid (“NDA”) with one or more aromatic diols (e.g., hydroquinone (“HQ”), 4,4′-biphenol (“BP”), etc.) to form ester repeating units. HQ constitutes a significant portion of the aromatic diols employed in most commercial liquid crystalline polymers. Conventionally, HQ has been produced from crude oil through a catalytic cracking process. Recently, however, a need for a more carbon neutral approach has been sought. To be carbon neutral, a company must remove the same amount of carbon dioxide that it is emitting into the atmosphere to achieve a net-zero carbon emissions. A carbon negative company, on the other hand, removes more carbon from the atmosphere than it releases. In view of the significant efforts across the globe of companies to become carbon neutral or carbon negative, a need exists for a process for producing biphenols and liquid crystalline polymers in a more sustainable way without significantly altering the properties of such polymers.

### SUMMARY OF THE INVENTION

In accordance with one embodiment of the present invention, a method for forming a bio-dihydroxyphenol monomer for use in a liquid crystalline polymer is disclosed. The method comprises providing a bio-dialkylated benzene that is derived from bio-naphtha; oxidizing the bio-dialkylated benzene to form a bio-dialkylated benzene hyperperoxide radical; and subjecting the bio-dialkylated benzene hyperperoxide radical to acid decomposition to form the bio-dihydroxyphenol.

In accordance with another embodiment of the present invention, a bio-liquid crystalline polymer is disclosed that comprises repeating units derived from one or more dihydroxyphenols, optional repeating units derived from one or more hydroxycarboxylic acids, optional repeating units derived from one or more dicarboxylic acids, and optional repeating units derived from one or more biphenols. The one or more dihydroxyphenols include a bio-hydroquinone derived from bio-naphtha.

Other features and aspects of the present invention are set forth in greater detail below.

### DETAILED DESCRIPTION

It is to be understood by one of ordinary skill in the art that the present discussion is a description of exemplary embodiments only, and is not intended as limiting the broader aspects of the present invention.

Generally speaking, the present disclosure is directed to a technique for forming a bio-based dihydroxyphenol monomer that is derived from bio-naphtha and a bio-liquid crystalline polymer (“bio-LCP”) formed therefrom. Ultimately, the bio-LCP and compositions formed from such polymers can be certified based on the ISCC+ standard set forth by the International Sustainability and Carbon Certification (“ISCC”) system. The ISCC is a globally applicable sustainability certification system and covers all sustainable feedstocks, including agricultural and forestry biomass, circular and bio-based materials and renewables. The ISCC follows a mass balance approach in which the renewable content of the polymer can be verified. In mass balance, renewable feedstock is attributed to selected products, according to their individual formulation taking into account all yields and losses. Only raw materials used as feedstock (but not for energy) for the production are considered for mass balancing. The key criteria used for applying the mass balance approach include feedstock qualification, chain of custody, and product claims. The mass balance approach makes it possible to track the amount and sustainability characteristics of recycled and/or bio-based feedstocks in the value chain and attribute it to the final product in a verifiable manner. In one embodiment, the liquid crystalline polymer described herein can be made from carbon negative or carbon neutral components under the mass balance approach.

Various embodiments of the present invention will now be described in more detail.

### 1. Bio-Dihydroxyphenols

As noted above, the bio-dihydroxyphenols of the present disclosure are derived from “bio-naphtha.” The term “bio-naphtha” generally refers to naphtha produced from a renewable source. Bio-naphtha is a hydrocarbon composition that primarily includes paraffins that can be converted into a bio-aromatic compound (e.g., bio-benzene). The hydrocarbon content typically has from 8 to 24 carbons, and in some cases from 10 to 18 carbons. To form bio-naphtha, a bio-distillate feedstock is typically provided that includes a complex mixture of natural occurring fats and/or oils, such as plant-based fats and oils (e.g., cotton, coconut, corn, palm, peanut, linseed, rice, rapeseed, olive, soybean, sunflower, linola, tallow, tall, castor, etc.) and animal-based fats and oils (e.g., butter or milk fat). The feedstock may be provided in an unused (virgin) state and/or obtained from a waste product, such as cooking oils, inedible highly saturated oils, waste food oils, by-products of the refining of vegetable oils, and mixtures thereof. Natural fats and oils primarily include triglycerides and to some extent of free fatty acids (FFA). Many different types of triglycerides are produced in nature, either from vegetable as from animal origin. Fatty acids in fats & oils are found esterified to glycerol (triacylglycerol). The acyl-group is a long-chain (C₁₂-C₂₂) hydrocarbon with a carboxyl-group at the end that is generally esterified with glycerol. Fats and oils are characterized by the chemical composition and structure of its fatty acid moiety. The fatty acid moiety can be saturated or contain one or more double bonds. The aforementioned sources of sources of fats and/or oils may include fatty acids, such as saturated fatty acids (e.g., caproic, caprylic, capric, lauric, myristic, palmitic, margaric, stearic, arachidic, behenic, lignoceric, etc.) and/or unsaturated fatty acids (e.g., myristoleic, palmitoleic, heptadecenoic, oleic, linoleic, linolenic, gadolenic, riconoleic, rosin, etc.). Tall oil, for example, contains myristic acid, stearic acid, arachidic acid, oleic acid, linoleic acid, and gadolenic acid.

Bio-distillate feedstocks may be classified based on their free fatty acid (FFA) content as follows: refined oils, such as soybean or refined canola oils (FFA<1.5%); low free fatty acid yellow greases and animal fats (FFA<4%); and high free fatty acid greases and animal fats (FFA>20%). In certain cases, it may be desired to subject the feedstock to a refining treatment (e.g., physical refining, chemical refining, etc.) to remove a major part of the non-triglyceride and non-fatty acid components. Physical refining, for example, can remove the FFA, as well as the unsaponifiables and other impurities by steam stripping, thus eliminating the production of soapstock and keeping neutral oil loss to a minimum. However, degumming pretreatments of the crude fats and oils may be required to remove those impurities that darken or otherwise cause a poor-quality product when heated to the temperature required for steam distillation. A degumming process may include treatment of crude oils, with water, salt solutions, enzymes, caustic soda, or diluted acids such as phosphoric, citric or maleic to remove phosphatides, waxes, pro-oxidants and other impurities. The degumming processes convert the phosphatides to hydrated gums, which are insoluble in oil and readily separated as a sludge by settling, filtering or centrifugal action.

The optionally refined oil may contain an unsaturated or substantially unsaturated, liquid or substantially liquid triglyceride part (phase L) and a saturated or substantially saturated, solid or substantially solid triglyceride part (phase S). The solid phase S may thereafter be transformed into linear or substantially linear paraffins as the “bio-naphtha.” More particularly, the refined oil can be fractioned into the phases L and S by a fractional crystallization method that includes a controlled cooling down during which the triglycerides of the complex mixture with substantially saturated acyl-moieties crystallize and precipitate from the mixture forming the phase S, while the triglycerides with substantially unsaturated acyl-moieties remain liquid forming the phase L, both phases being then separated by simple filtration or decantation or centrifugation. In one embodiment, fractionation may be “dry fractionation” or “dry winterization”, which is the removal of solids by controlled crystallization and separation techniques involving the use of solvents or dry processing (sometimes also referred to as dewaxing). The fractionation process has two main stages, the first being the crystallization stage. Crystals grow when the temperature of the molten fat and oil or its solution is lowered, and their solubility at the final or separation temperature determines the triglycerides composition of the crystals formed as well as their mother liquor. The separation process is the second step of fractionation. Several options may be employed, such as vacuum filters, centrifugal separators, conical screen-scroll centrifuges, hydraulic presses, membrane filter presses, or decanters. Fractionation may also been carried out in presence of solvents, such as paraffins, alkyl-acetates, ethers, ketones, alcohols or chlorinated hydrocarbons.

Once obtained, the phase S may converted into linear or substantially linear paraffins as bio-naphtha through known processes, such as thermal decarboxylation (e.g., using a soap feedstock obtained from chemically refining fats and oils), catalytic decarboxylation (e.g., using fatty acid feedstock obtained by physically refining oils and fats), and catalytic hydrodeoxygenation (using triglyceride and/or fatty acid feedstock). Hydrodeoxygenation is often employed and ultimately involves the removal of oxygen atoms from the fats and oils. Hydrodeoxygenation is preferentially done in continuous fixed bed reactors, continuous stirred tank reactors or slurry type reactors containing a solid catalyst. The catalyst may include, for instance, Ni, Mo, Co or mixtures, such as NiW, NiMo, CoMo, NiCoW, NiCoMo, NiMoW and CoMoW oxides or sulphides as catalytic active phase, preferably supported on high surface area carbon, alumina, silica, titania or zirconia or group 10 (Ni, Pt or Pd) or group 11 (Cu or Ag) metals or alloy mixtures supported on high surface area carbon, magnesia, zinc-oxide, spinels (Mg₂Al₂O₄, ZnAl₂O₄), perovskites (BaTiO₃, ZnTiO₃), calcium silicates (e.g., xonotlite), alumina, silica or mixtures of the latter. It is preferred that the support for the catalytic active phase exhibit low acidity, preferable neutral or basic in order to avoid hydro-isomerization reactions that would result in branched paraffins and cracking. Hydrodeoxygenation may be carried out at a temperature from about 200° C. to about 500° C., and in some embodiments, from about 280° C. to about 400° C., under a pressure of from about 1 MPa to about 10 MPa and with a hydrogen to refined oils ratio of from about 100 to about 2000, and in some embodiments, from about 350 to about 1500.

Regardless of the manner in which it is formed, the resulting feedstock containing bio-naphtha may be subjected to a steam cracking process to obtain a bio-aromatic compound (e.g., bio-benzene). Steam crackers are complex industrial facilities that can be divided into three main zones, each of which has several types of equipment with very specific functions: (i) the hot zone including: pyrolysis or cracking furnaces, quench exchanger and quench ring, the columns of the hot separation train (ii) the compression zone including: a cracked gas compressor, purification and separation columns, dryers and (iii) the cold zone including: the cold box, de-methanizer, fractionating columns of the cold separation train, the C₂ and C₃ converters, the gasoline hydrostabilization reactor. Hydrocarbon cracking may be carried out in tubular reactors in direct-fired heaters (furnaces). Various tube sizes and configurations can be used, such as coiled tube, U-tube, or straight tube layouts. Each furnace consists of a convection zone in which the waste heat is recovered and a radiant zone in which pyrolysis takes place. The feedstock-steam mixture is preheated in the convection zone to about 530-650° C. or the feedstock is preheated in the convection section and subsequently mixed with dilution steam before it flows over to the radiant zone, where pyrolysis takes place at temperatures varying from 750 to 950° C. The steam/feedstock (the steam/[hydrocarbon feedstock]) weight ratio may be from about 0.2 to about 1.0 kg/kg. For steam cracking furnaces, the severity can be modulated by: temperature, residence time, total pressure and partial pressure of hydrocarbons. Effluent from the pyrolysis furnaces contains unreacted feedstock, olefins (mainly ethylene and propylene), hydrogen, methane, a mixture of C₄ (primarily isobutylene and butadiene), aromatics in the C₆ to C₈ range, ethane, propane, di-olefins (acetylene, methyl acetylene, propadiene), and heavier hydrocarbons that boil in the temperature range of fuel oil. This cracked gas is rapidly quenched to 338-510° C. to stop the pyrolysis reactions, minimize consecutive reactions and to recover the sensible heat in the gas by generating high-pressure steam in parallel transfer-line heat exchangers (TLEs).

The resulting mixed hydrocarbon feed may then be supplied to a “dearomatization unit”, which is a refinery unit for the separation of aromatic hydrocarbons (e.g., bio-benzene). Such dearomatization processes are described in Folkins (2000) Benzene, Ullmann's Encyclopedia of Industrial Chemistry. One particular method to separate aromatic hydrocarbons from a mixture of aromatic and aliphatic hydrocarbons is solvent extraction, such as described in WO 2012/135111, which is incorporated herein by reference thereto. The preferred solvents used in aromatic solvent extraction are sulfolane, tetraethylene glycol and N-methylpyrolidone which are commonly used solvents in commercial aromatics extraction processes. These species are often used in combination with other solvents or other chemicals (sometimes called co-solvents) such as water and/or alcohols. Non-nitrogen containing solvents such as sulfolane are particularly preferred. Solvent extraction of heavy aromatics is described in the art, see e.g. U.S. Pat. No. 5,880,325, which is incorporated herein in its entirety by reference thereto. Alternatively, other known methods than solvent extraction, such as molecular sieve separation or separation based on boiling point, can be applied for the separation of heavy aromatics in a dearomatization process.

Once obtained, the resulting bio-benzene may thereafter be converted into a bio-dialkylbenzene. For example, one suitable process involves an alkylation reaction using propylene and bio-benzene feedstocks. A typical propylene feedstock may be an almost pure polymer grade material or can contain significant amounts of propane, as typically found in refinery-grade propylene. A typical bio-benzene feedstock may contain benzene (99.9 wt.-% min.) and toluene (0.05 wt.-% min). Alkylation reactors may be operated in the vapor phase, liquid-phase or mixed-phase. At the lower temperatures of the liquid phase operation, xylene impurities are not produced and a cumene product of superior quality is produced. The temperature is typically from about 100° C. to about 310° C. and the pressure is typically from about 8 to 50 bar. The alkylation reactor may contain an effective amount of an alkylation catalyst, such as solid acid catalysts (e.g., solid oxide zeolite). Examples are zeolite beta, zeolite X, zeolite Y, mordenite, faujasite, zeolite omega, UZM-8, MCM-22, MCM-36, MCM-49 and MCM-56.

In the alkylation reactor, the bio-benzene is dialkylated with two equivalents of propylene to form a bio-dialkylated benzene (e.g., 1,4-diisopropylbenzene for producing bio-hydroquinone or 1,3-diisopropylbenzene for producing resorcinol). The bio-dialkylated benzene may be initially oxidized to form a bio-dialkylated benzene hyperperoxide radical (e.g., 1,4-diisopropylbenzene monohydroxyperoxide, 1,4-diisopropylbenzene dihydroxyperoxide, 1,3-diisopropylbenzene monohydroxyperoxide, 1,3-diisopropylbenzene dihydroxyperoxide, etc.). This may occur through oxidation of the dialkylated benzene in an alkaline medium in which the hydroperoxide product is stable. Oxidation with air or oxygen may occur at mildly elevated temperatures of about 70° C. to about 130° C. In addition to the dialkylated benzene, monohydroxyperoxide radical, and dihydroxyperoxide radical, these oxidation reaction mixtures may also contain other peroxide radicals (e.g., p-2-hydroxy-2-propyl-α,α-dimethylbenzylhydroperoxide), other alcohols, olefins, tar materials, etc. These oxidation reaction mixtures may themselves be used as a starting material for the subsequent reaction steps. Alternatively, to convert the other peroxide radicals contained in these mixtures to a dihydroxyperoxide radical, the above-mentioned oxidation reaction mixtures can be used after they are further oxidized by an oxidation agent (e.g., hydrogen peroxide). Furthermore, these types of oxidation reaction mixtures may be used after a portion or all of the unreacted starting materials or by-products are separated and removed therefrom.

Regardless of the particular starting material(s), the bio-dialkylated benzene hyperperoxide radical then undergoes acid decomposition in the presence of a solvent to form a bio-dihydroxyphenol (e.g., bio-hydroquinone, bio-resorcinol, etc.). A solvent that is capable of dissolving both the dihydroxyphenol and dihydroxyperoxide radical is particularly suitable, including organic solvents, such as ketones having approximately 6 through 10 carbon atoms (e.g., methyl isobutyl ketone, diisopropyl ketones, diisobutyl ketone, etc.); aromatic hydrocarbons, and so forth. An acid catalyst may also be employed, such as a water-soluble acid (e.g., sulfuric acid, phosphoric acid, perchloric acid, etc.) or a solid acid (e.g., silica-alumina, silica-magnesia, cation exchange resins, etc.). The reaction temperature is advantageously carried out at a temperature of about 20° C. to about 90° C., and in some embodiments, from about 50° C. to about 80° C., for a period of time of from about 5 to about 60 minutes, and in some embodiments, from about 15 to about 30 minutes. The acid decomposition reaction can be terminated by removal of the acid catalyst, such as by contacting the reaction mixture with an aqueous solution of a neutral salt such as, for example, an aqueous solution of sodium sulfate, potassium sulfate, sodium chloride, sodium phosphate, or ammonium sulfate. In this manner, the acid may be extracted from the reaction mixture to the aqueous solution layer. Upon removal of the acid, the resulting reaction mixture may still contain components other than the bio-dihydroxyphenols, such as solvents, acetone, water, and other by products. To remove these components from the reaction mixture and separate the bio-dihydroxyphenols, one or more distillation operations may be carried out as are well known in the art, such as described in more detail in U.S. Pat. No. 4,434,305, which is incorporated herein by reference thereto.

### II. Bio-Liquid Crystalline Polymers (“Bio-LCP”)

As indicated above, the resulting bio-dihydroxyphenols may be particularly suitable for use in forming a bio-based liquid crystalline polymer (“bio-LCP”). Bio-LCP generally contains aromatic repeating units derived from the bio-biphenol(s) described above. Examples of such dihydroxyphenols may include, for instance, hydroquinone, resorcinol, 2,7-dihydroxynaphthalene, 1,6-dihydroxynaphthalene, etc., as well as alkyl, alkoxy, aryl and halogen substituents thereof, and combinations thereof. Particularly suitable is bio-hydroquinone (“bio-HQ”). Desirably, all of the dihydroxyphenols employed in the liquid crystalline polymer are bio-dihydroxyphenols derived from bio-naphtha. However, this is by no means required and a portion of such dihydroxyphenols may also be derived from traditional fossil fuel sources (e.g., petroleum) as is known in the art. Regardless, the repeating units derived from dihydroxyphenols (e.g., bio-dihydroxyphenols) typically constitute from about 1 mol. % to about 50 mol. %, in some embodiments from about 5 mol. % to about 40 mol. %, and in some embodiments, from about 10 mol. % to about 35 mol. % of the polymer. In one particular embodiment, for instance, the liquid crystalline polymer may contain repeating units derived from bio-HQ in an amount from 6 mol. % to about 40 mol. %, in some embodiments from about 7 mol. % to about 35 mol. %, and in some embodiments, from about 10 mol. % to about 30 mol. %.

The bio-LCP may also contain aromatic repeating units derived from other types of aromatic diols, such as biphenols, including 4,4′-biphenol, 3,3′-dihydroxybiphenyl, 3,4′-dihydroxybiphenyl, 4,4′-dihydroxybiphenyl ether, bis(4-hydroxyphenyl)ethane, etc., as well as alkyl, alkoxy, aryl and halogen substituents thereof, and combinations thereof. When employed, repeating units derived from such other aromatic diols (e.g., BP) typically constitute from about 1 mol. % to about 50 mol. %, in some embodiments from about 5 mol. % to about 40 mol. %, and in some embodiments, from about 10 mol. % to about 35 mol. % of the polymer.

Aromatic hydroxycarboxylic acids may also be employed, such as hydroxybenzoic acids (e.g., 4-hydroxybenzoic acid, 4′-hydroxyphenyl-4-benzoic acid, 3′-hydroxyphenyl-4-benzoic acid, 4′-hydroxyphenyl-3-benzoic acid, etc.); hydroxynaphthenic acids (e.g., 2-hydroxy-6-naphthoic acid (“HNA”); 2-hydroxy-5-naphthoic acid; 3-hydroxy-2-naphthoic acid; 2-hydroxy-3-naphthoic acid, etc.), as well as alkyl, alkoxy, aryl and halogen substituents thereof. Particularly suitable is bio-4-hydroxybenzoic acid (“HBA”) and 2-hydroxy-6-naphthoic acid. When employed, the repeating units derived from hydroxybenzoic acids typically constitute from about 20 mol. % to about 85 mol. %, in some embodiments from about 30 mol. % to about 80 mol. %, and in some embodiments, from about 40 mol. % to 75 mol. % of the polymer and the repeating units derived from hydroxynaphthenic acids typically constitute from about 1 mol. % to about 50 mol. %, in some embodiments from about 2 mol. % to about 40 mol. %, and in some embodiments, from about 5 mol. % to 30 mol. % of the polymer. In one particular embodiment, for instance, the liquid crystalline polymer may contain repeating units derived from HBA in an amount from 40 mol. % to about 85 mol. %, in some embodiments from about 45 mol. % to about 82 mol. %, and in some embodiments, from about 50 mol. % to about 80 mol. %.

Aromatic dicarboxylic repeating units may also be employed that are derived from aromatic dicarboxylic acids, such as terephthalic acid, isophthalic acid, 2,6-naphthalenedicarboxylic acid, diphenyl ether-4,4′-dicarboxylic acid, 1,6-naphthalenedicarboxylic acid, 2,7-naphthalenedicarboxylic acid, 4,4′-dicarboxybiphenyl, bis(4-carboxyphenyl)ether, bis(4-carboxyphenyl)butane, bis(4-carboxyphenyl)ethane, bis(3-carboxyphenyl)ether, bis(3-carboxyphenyl)ethane, etc., as well as alkyl, alkoxy, aryl and halogen substituents thereof, and combinations thereof. Particularly suitable aromatic dicarboxylic acids may include, for instance, terephthalic acid (“TA”), isophthalic acid (“IA”), and 2,6-naphthalenedicarboxylic acid (“NDA”). When employed, repeating units derived from aromatic dicarboxylic acids (e.g., IA, TA, and/or NDA) typically constitute from about 1 mol. % to about 50 mol. %, in some embodiments from about 5 mol. % to about 40 mol. %, and in some embodiments, from about 10 mol. % to about 35 mol. % of the polymer. Repeating units may also be employed, such as those derived from aromatic amides (e.g., acetaminophen (“APAP”)) and/or aromatic amines (e.g., 4-aminophenol (“AP”), 3-aminophenol, 1,4-phenylenediamine, 1,3-phenylenediamine, etc.). When employed, repeating units derived from aromatic amides (e.g., APAP) and/or aromatic amines (e.g., AP) typically constitute from about 0.1 mol. % to about 20 mol. %, in some embodiments from about 0.5 mol. % to about 15 mol. %, and in some embodiments, from about 1 mol. % to about 10% of the polymer. It should also be understood that various other monomeric repeating units may be incorporated into the polymer. For instance, in certain embodiments, the polymer may contain one or more repeating units derived from non-aromatic monomers, such as aliphatic or cycloaliphatic hydroxycarboxylic acids, dicarboxylic acids, diols, amides, amines, etc. Of course, in other embodiments, the polymer may be “wholly aromatic” in that it lacks repeating units derived from non-aromatic (e.g., aliphatic or cycloaliphatic) monomers.

Regardless of the particular constituents and nature of the polymer, the bio-LCP may be prepared by initially introducing the aromatic monomer(s) used to form the repeating units (e.g., aromatic hydroxycarboxylic acid, aromatic dicarboxylic acid, etc.) and/or other repeating units (e.g., aromatic diol, aromatic amide, aromatic amine, etc.) into a reactor vessel to initiate a polycondensation reaction. The particular conditions and steps employed in such reactions are well known, and may be described in more detail in U.S. Pat. No. 4,161,470 to Calundann; U.S. Pat. No. 5,616,680 to Linstid, III, et al.; U.S. Pat. No. 6,114,492 to Linstid, III, et al.; U.S. Pat. No. 6,514,611 to Shepherd, et al.; and WO 2004/058851 to Waggoner. The vessel employed for the reaction is not especially limited, although it is typically desired to employ one that is commonly used in reactions of high viscosity fluids. Examples of such a reaction vessel may include a stirring tank-type apparatus that has an agitator with a variably-shaped stirring blade, such as an anchor type, multistage type, spiral-ribbon type, screw shaft type, etc., or a modified shape thereof. Further examples of such a reaction vessel may include a mixing apparatus commonly used in resin kneading, such as a kneader, a roll mill, a Banbury mixer, etc.

If desired, the reaction may proceed through the acetylation of the monomers as known the art. This may be accomplished by adding an acetylating agent (e.g., acetic anhydride) to the monomers. Acetylation is generally initiated at temperatures of about 90° C. During the initial stage of the acetylation, reflux may be employed to maintain vapor phase temperature below the point at which acetic acid byproduct and anhydride begin to distill. Temperatures during acetylation typically range from between 90° C. to 150° C., and in some embodiments, from about 110° C. to about 150° C. If reflux is used, the vapor phase temperature typically exceeds the boiling point of acetic acid, but remains low enough to retain residual acetic anhydride. For example, acetic anhydride vaporizes at temperatures of about 140° C. Thus, providing the reactor with a vapor phase reflux at a temperature of from about 110° C. to about 130° C. is particularly desirable. To ensure substantially complete reaction, an excess amount of acetic anhydride may be employed. The amount of excess anhydride will vary depending upon the particular acetylation conditions employed, including the presence or absence of reflux. The use of an excess of from about 1 to about 10 mole percent of acetic anhydride, based on the total moles of reactant hydroxyl groups present is not uncommon.

Acetylation may occur in in a separate reactor vessel, or it may occur in situ within the polymerization reactor vessel. When separate reactor vessels are employed, one or more of the monomers may be introduced to the acetylation reactor and subsequently transferred to the polymerization reactor. Likewise, one or more of the monomers may also be directly introduced to the reactor vessel without undergoing pre-acetylation.

In addition to the monomers and optional acetylating agents, other components may also be included within the reaction mixture to help facilitate polymerization. For instance, a catalyst may be optionally employed, such as metal salt catalysts (e.g., magnesium acetate, tin(I) acetate, tetrabutyl titanate, lead acetate, sodium acetate, potassium acetate, etc.) and organic compound catalysts (e.g., N-methylimidazole). Such catalysts are typically used in amounts of from about 50 to about 500 parts per million based on the total weight of the recurring unit precursors. When separate reactors are employed, it is typically desired to apply the catalyst to the acetylation reactor rather than the polymerization reactor, although this is by no means a requirement.

The reaction mixture is generally heated to an elevated temperature within the polymerization reactor vessel to initiate melt polycondensation of the reactants. Polycondensation may occur, for instance, within a temperature range of from about 200° C. to about 400° C. For instance, one suitable technique for forming the aromatic polyester may include charging precursor monomers and acetic anhydride into the reactor, heating the mixture to a temperature of from about 90° C. to about 150° C. to acetylize a hydroxyl group of the monomers (e.g., forming acetoxy), and then increasing the temperature to from about 200° C. to about 400° C. to carry out melt polycondensation. As the final polymerization temperatures are approached, volatile byproducts of the reaction (e.g., acetic acid) may also be removed so that the desired molecular weight may be readily achieved. The reaction mixture is generally subjected to agitation during polymerization to ensure good heat and mass transfer, and in turn, good material homogeneity. The rotational velocity of the agitator may vary during the course of the reaction, but typically ranges from about 10 to about 100 revolutions per minute (“rpm”), and in some embodiments, from about 20 to about 80 rpm. To build molecular weight in the melt, the polymerization reaction may also be conducted under vacuum, the application of which facilitates the removal of volatiles formed during the final stages of polycondensation. The vacuum may be created by the application of a suctional pressure, such as within the range of from about 5 to about 30 pounds per square inch (“psi”), and in some embodiments, from about 10 to about 20 psi.

Following melt polymerization, the molten polymer may be discharged from the reactor, typically through an extrusion orifice fitted with a die of desired configuration, cooled, and collected. Commonly, the melt is discharged through a perforated die to form strands that are taken up in a water bath, pelletized and dried. In some embodiments, the melt polymerized polymer may also be subjected to a subsequent solid-state polymerization method to further increase its molecular weight. Solid-state polymerization may be conducted in the presence of a gas (e.g., air, inert gas, etc.). Suitable inert gases may include, for instance, include nitrogen, helium, argon, neon, krypton, xenon, etc., as well as combinations thereof. The solid-state polymerization reactor vessel can be of virtually any design that will allow the polymer to be maintained at the desired solid-state polymerization temperature for the desired residence time. Examples of such vessels can be those that have a fixed bed, static bed, moving bed, fluidized bed, etc. The temperature at which solid-state polymerization is performed may vary, but is typically within a range of from about 200° C. to about 400° C. The polymerization time will of course vary based on the temperature and target molecular weight. In most cases, however, the solid-state polymerization time will be from about 2 to about 12 hours, and in some embodiments, from about 4 to about 10 hours.

Once formed, the resulting bio-LCP may have a “bio-content” of about 1 wt. % to 100 wt. %, in some embodiments about 2 wt. % to about 90 wt. %, in some embodiments from about 5 wt. % to about 70 wt. %, and in some embodiments, from about 10 wt. % to about 60 wt. % based on the total weight of monomers (repeating units) employed in the polymer. As used herein, the term “bio-content” generally refers to the weight percentage of monomers (repeating units) that are derived from bio-naphtha. Thus, it should be understood that this weight percentage may include the bio-biphenols described herein (e.g., bio-hydroquinone), as well as other monomer components that may also be derived from bio-naphtha, such as bio-4-hydroxybenzoic acid (“bio-HBA”), bio-terephthalic acid (“bio-TA”), bio-isophthalic acid (“bio-IA”), bio-4,4′-biophenol (“bio-BP”), bio-2-hydroxy-6-naphthoic acid (“bio-HNA”), bio-2,6-naphthalenedicarboxylic acid (“bio-NDA”), bio-4-aminophenol (“bio-AP”), bio-acetaminophen (“bio-APAP”), etc. Despite containing such a high bio-content, the resulting bio-LCP is still capable of exhibiting similar properties to liquid crystalline polymers formed from traditional fossil fuel sources. Namely, the bio-LCP is still considered “thermotropic” to the extent that it can possess a rod-like structure and exhibit a crystalline behavior in its molten state (e.g., thermotropic nematic state). The bio-LCP also typically has a high melting temperature, such as of from about 280° C. to about 400° C., in some embodiments from about 290° C. to about 380° C., and in some embodiments from about 300° C. to about 350° C. The melting temperature may be determined as is well known in the art using differential scanning calorimetry (“DSC”), such as determined by ISO 11357-3:2018.

### III. Polymer Composition

The bio-LCP may be used in neat form (i.e., composition containing 100 wt. % of bio-LCP(s)) or blended with other components to form a polymer composition. In such embodiments, bio-LCP(s) typically constitute from about 10 wt. % to about 90 wt. %, in some embodiments from about 20 wt. % to about 80 wt. %, in some embodiments from about 25 wt. % to about 70 wt. %, and in some embodiments, from about 30 wt. % to about 60 wt. % of the polymer composition. In such embodiments, other additives likewise constitute from about 10 wt. % to about 90 wt. %, in some embodiments from about 20 wt. % to about 80 wt. %, in some embodiments from about 30 wt. % to about 75 wt. %, and in some embodiments, from about 40 wt. % to about 70 wt. % of the polymer composition. If desired, the other additives may also be derived from a sustainable source, such as recycled materials, renewable materials, bio-based materials, etc. For example, the total “sustainable content” of the polymer composition is typically from about 5 wt. % to 100 wt. %, in some embodiments from about 10 wt. % to about 90 wt. %, and in some embodiments, from about 20 wt. % to about 80 wt. % based on the total weight of the composition. The term “sustainable content” generally refers to the weight percentage of components that are derived from a sustainable source. For a composition containing only bio-LCP, for example, the “sustainable content” is the same as the “bio-content” (weight percentage of monomers derived from bio-naphtha). For compositions containing bio-LCP, other sustainable materials (e.g., recycled materials), non-sustainable materials (e.g., fossil fuel-based materials, virgin materials, etc.), the “sustainable content” can be determined as follows:

$$\frac{\begin{matrix}( Weight of Bio - LCP ) * ( “ Bio - Content ” ) + \\ ( Weight of Other Sustainable Materials )\end{matrix}}{Weight of Polymer Composition}$$

Various examples of other additives that can be used in the polymer composition are set forth below.

### A. Mineral Filler

The polymer composition may optionally contain one or more mineral fillers. When employed, such mineral filler(s) typically constitute from about 1 wt. % to about 50 wt. %, in some embodiments from about 2 wt. % to about 45 wt. %, and in some embodiments, from about 5 wt. % to about 40 wt. % of the polymer composition. The nature of the mineral filler(s) employed in the polymer composition may vary, such as mineral particles, mineral fibers (or “whiskers”), etc., as well as blends thereof. Typically, the mineral filler(s) employed in the polymer composition have a certain hardness value to help improve the mechanical strength, adhesive strength, and surface properties of the composition. For instance, the hardness values may be about 2.0 or more, in some embodiments about 2.5 or more, in some embodiments about 3.0 or more, in some embodiments from about 3.0 to about 11.0, in some embodiments from about 3.5 to about 11.0, and in some embodiments, from about 4.5 to about 6.5 based on the Mohs hardness scale.

Any of a variety of different types of mineral particles may generally be employed in the polymer composition, such as those formed from a natural and/or synthetic silicate mineral, such as talc, mica, silica (e.g., amorphous silica), alumina, halloysite, kaolinite, illite, montmorillonite, vermiculite, palygorskite, pyrophyllite, calcium silicate, aluminum silicate, wollastonite, etc.; sulfates; carbonates; phosphates; fluorides, borates; and so forth. Particularly suitable are particles include talc, calcium carbonate (CaCO₃), copper carbonate hydroxide (Cu₂CO₃(OH)₂); calcium fluoride (CaFl₂); calcium pyrophosphate ((Ca₂P₂O₇), anhydrous dicalcium phosphate (CaHPO₄), hydrated aluminum phosphate (AlPO₄·2H₂O); silica (SiO₂), potassium aluminum silicate (KAlSi₃O₈), copper silicate (CuSiO₃·H₂O); calcium borosilicate hydroxide (Ca₂B₅SiO₉(OH)₅); alumina (AlO₂); calcium sulfate (CaSO₄), barium sulfate (BaSO₄), talc, mica, and so forth, as well as combinations thereof. Talc, mica, calcium carbonate, and barium sulfate are particularly suitable. Any form of mica may generally be employed, including, for instance, muscovite (KAl₂(AlSi₃)O₁₀(OH)₂), biotite (K(Mg,Fe)₃(AlSi₃)O₁₀(OH)₂), phlogopite (KMg₃(AlSi₃)O₁₀(OH)₂), lepidolite (K(Li,Al)₂₋₃(AlSi₃)O₁₀(OH)₂), glauconite (K,Na)(Al,Mg,Fe)₂(Si,Al)₄O₁₀(OH)₂), etc.

In certain embodiments, the mineral particles, such as barium sulfate and/or calcium sulfate particles, may have a shape that is generally granular or nodular in nature. In such embodiments, the particles may have a median size (e.g., diameter) of from about 0.5 to about 20 micrometers, in some embodiments from about 1 to about 15 micrometers, in some embodiments from about 1.5 to about 10 micrometers, and in some embodiments, from about 2 to about 8 micrometers, such as determined using laser diffraction techniques in accordance with ISO 13320:2009 (e.g., with a Horiba LA-960 particle size distribution analyzer). In other embodiments, it may also be desirable to employ flake-shaped mineral particles, such as mica particles, that have a relatively high aspect ratio (e.g., average diameter divided by average thickness), such as about 4 or more, in some embodiments about 8 or more, and in some embodiments, from about 10 to about 500. In such embodiments, the average diameter of the particles may, for example, range from about 5 micrometers to about 200 micrometers, in some embodiments from about 8 micrometers to about 150 micrometers, and in some embodiments, from about 10 micrometers to about 100 micrometers. The average thickness may likewise be about 2 micrometers or less, in some embodiments from about 5 nanometers to about 1 micrometer, and in some embodiments, from about 20 nanometers to about 500 nanometers such as determined using laser diffraction techniques in accordance with ISO 13320:2009 (e.g., with a Horiba LA-960 particle size distribution analyzer). The mineral particles may also have a narrow size distribution. That is, at least about 70% by volume of the particles, in some embodiments at least about 80% by volume of the particles, and in some embodiments, at least about 90% by volume of the particles may have a size within the ranges noted above.

Suitable mineral fibers may likewise include those that are derived from silicates, such as neosilicates, sorosilicates, inosilicates (e.g., calcium inosilicates, such as wollastonite; calcium magnesium inosilicates, such as tremolite; calcium magnesium iron inosilicates, such as actinolite; magnesium iron inosilicates, such as anthophyllite; etc.), phyllosilicates (e.g., aluminum phyllosilicates, such as palygorskite), tectosilicates, etc.; sulfates, such as calcium sulfates (e.g., dehydrated or anhydrous gypsum); mineral wools (e.g., rock or slag wool); and so forth. Particularly suitable are fibers having the desired hardness value, including fibers derived from inosilicates, such as wollastonite (Mohs hardness of 4.5 to 5.0), which are commercially available from Nyco Minerals under the trade designation Nyglos® (e.g., Nyglos® 4 W or Nyglos® 8). The mineral fibers may have a median width (e.g., diameter) of from about 1 to about 35 micrometers, in some embodiments from about 2 to about 20 micrometers, in some embodiments from about 3 to about 15 micrometers, and in some embodiments, from about 7 to about 12 micrometers. The mineral fibers may also have a narrow size distribution. That is, at least about 60% by volume of the fibers, in some embodiments at least about 70% by volume of the fibers, and in some embodiments, at least about 80% by volume of the fibers may have a size within the ranges noted above. In addition to possessing the size characteristics noted above, the mineral fibers may also have a relatively high aspect ratio (average length divided by median width) to help further improve the mechanical properties and surface quality of the resulting polymer composition. For example, the mineral fibers may have an aspect ratio of from about 2 to about 100, in some embodiments from about 2 to about 50, in some embodiments from about 3 to about 20, and in some embodiments, from about 4 to about 15. The volume average length of such mineral fibers may, for example, range from about 1 to about 200 micrometers, in some embodiments from about 2 to about 150 micrometers, in some embodiments from about 5 to about 100 micrometers, and in some embodiments, from about 10 to about 50 micrometers.

### B. Fibrous Filler

A fibrous filler may also be employed in the polymer composition. The fibrous filler typically includes fibers having a high degree of tensile strength relative to their mass. For example, the ultimate tensile strength of the fibers (determined in accordance with ASTM D2101) is typically from about 1,000 to about 15,000 Megapascals (“MPa”), in some embodiments from about 2,000 MPa to about 10,000 MPa, and in some embodiments, from about 3,000 MPa to about 6,000 MPa. To help maintain the desired properties, such high strength fibers may be formed from materials that are generally insulative in nature, such as glass, ceramics (e.g., alumina or silica), aramids (e.g., Kevlar® marketed by E. I. du Pont de Nemours, Wilmington, Del.), polyolefins, polyesters, etc. Glass fibers are particularly suitable, such as E-glass, A-glass, C-glass, D-glass, AR-glass, R-glass, S1-glass, S2-glass, etc. If desired, all or a portion of such fibers may be recycled.

Although the fibers employed in the fibrous filler may have a variety of different sizes, fibers having a certain aspect ratio can help improve the mechanical properties of the resulting polymer composition. That is, fibers having an aspect ratio (average length divided by nominal diameter) of from about 5 to about 50, in some embodiments from about 6 to about 40, and in some embodiments, from about 8 to about 25 are particularly beneficial. Such fibers may, for instance, have a weight average length of from about 100 to about 800 micrometers, in some embodiments from about 120 to about 500 micrometers, in some embodiments, from about 150 to about 350 micrometers, and in some embodiments, from about 200 to about 300 micrometers. The fibers may likewise have a nominal diameter of about 6 to about 35 micrometers, and in some embodiments, from about 9 to about 18 micrometers. The relative amount of the fibrous filler may also be selectively controlled to help achieve the desired mechanical and thermal properties. For example, the fibrous filler may constitute from about 1 wt. % to about 40 wt. %, in some embodiments from about 3 wt. % to about 30 wt. %, and in some embodiments, from about 5 wt. % to about 20 wt. % of the polymer composition.

### C. Impact Modifier

An impact modifier may also be employed in the polymer composition. For example, the impact modifier may be a polymer that contains an olefinic monomeric unit that derived from one or more α-olefins. Examples of such monomers include, for instance, linear and/or branched α-olefins having from 2 to 20 carbon atoms and typically from 2 to 8 carbon atoms. Specific examples include ethylene, propylene, 1-butene; 3-methyl-1-butene; 3,3-dimethyl-1-butene; 1-pentene; 1-pentene with one or more methyl, ethyl or propyl substituents; 1-hexene with one or more methyl, ethyl or propyl substituents; 1-heptene with one or more methyl, ethyl or propyl substituents; 1-octene with one or more methyl, ethyl or propyl substituents; 1-nonene with one or more methyl, ethyl or propyl substituents; ethyl, methyl or dimethyl-substituted 1-decene; 1-dodecene; and styrene. Particularly desired α-olefin monomers are ethylene and propylene. The olefin polymer may be in the form of a copolymer that contains other monomeric units as known in the art. For example, another suitable monomer may include a “(meth)acrylic” monomer, which includes acrylic and methacrylic monomers, as well as salts or esters thereof, such as acrylate and methacrylate monomers. Examples of such (meth)acrylic monomers may include methyl acrylate, ethyl acrylate, n-propyl acrylate, i-propyl acrylate, n-butyl acrylate, s-butyl acrylate, i-butyl acrylate, t-butyl acrylate, n-amyl acrylate, i-amyl acrylate, isobornyl acrylate, n-hexyl acrylate, 2-ethylbutyl acrylate, 2-ethylhexyl acrylate, n-octyl acrylate, n-decyl acrylate, methylcyclohexyl acrylate, cyclopentyl acrylate, cyclohexyl acrylate, methyl methacrylate, ethyl methacrylate, 2-hydroxyethyl methacrylate, n-propyl methacrylate, n-butyl methacrylate, i-propyl methacrylate, i-butyl methacrylate, n-amyl methacrylate, n-hexyl methacrylate, i-amyl methacrylate, s-butyl-methacrylate, t-butyl methacrylate, 2-ethylbutyl methacrylate, methylcyclohexyl methacrylate, cinnamyl methacrylate, crotyl methacrylate, cyclohexyl methacrylate, cyclopentyl methacrylate, 2-ethoxyethyl methacrylate, isobornyl methacrylate, etc., as well as combinations thereof. In one embodiment, for instance, the impact modifier may be an ethylene methacrylic acid copolymer (“EMAC”). When employed, the relative portion of the monomeric component(s) may be selectively controlled. The α-olefin monomer(s) may, for instance, constitute from about 55 wt. % to about 95 wt. %, in some embodiments from about 60 wt. % to about 90 wt. %, and in some embodiments, from about 65 wt. % to about 85 wt. % of the copolymer. Other monomeric components (e.g., (meth)acrylic monomers) may constitute from about 5 wt. % to about 35 wt. %, in some embodiments from about 10 wt. % to about 32 wt. %, and in some embodiments, from about 15 wt. % to about 30 wt. % of the copolymer.

Other suitable olefin copolymers may be those that are “epoxy-functionalized” in that they contain, on average, two or more epoxy functional groups per molecule. The copolymer may also contain an epoxy-functional monomeric unit. One example of such a unit is an epoxy-functional (meth)acrylic monomeric component. For example, suitable epoxy-functional (meth)acrylic monomers may include, but are not limited to, those containing 1,2-epoxy groups, such as glycidyl acrylate and glycidyl methacrylate. Other suitable epoxy-functional monomers include allyl glycidyl ether, glycidyl ethylacrylate, and glycidyl itoconate. Other suitable monomers may also be employed to help achieve the desired molecular weight. In one particular embodiment, for example, the copolymer may be a terpolymer formed from an epoxy-functional (meth)acrylic monomeric component, α-olefin monomeric component, and non-epoxy functional (meth)acrylic monomeric component. The copolymer may, for instance, be poly(ethylene-co-butylacrylate-co-glycidyl methacrylate). When employed, the epoxy-functional (meth)acrylic monomer(s) typically constitutes from about 1 wt. % to about 20 wt. %, in some embodiments from about 2 wt. % to about 15 wt. %, and in some embodiments, from about 3 wt. % to about 10 wt. % of the copolymer.

When employed, impact modifiers typically constitute from about 0.5 to about 60 parts, in some embodiments from about 1 to about 50 parts, and in some embodiments, from about 2 to about 30 parts by weight per 100 parts by weight of the liquid crystalline polymers employed in the composition. For example, impact modifiers may constitute from about 0.1 wt. % to about 30 wt. %, in some embodiments from about 0.5 wt. % to about 25 wt. %, and in some embodiments, from about 1 wt. % to about 20 wt. % of the polymer composition.

### D. Laser Activatable Additive

Although by no means required, the polymer composition may be “laser activatable” in the sense that it contains an additive that can be activated by a laser direct structuring (“LDS”) process. In such a process, the additive is exposed to a laser that causes the release of metals. The laser thus draws the pattern of conductive elements onto the part and leaves behind a roughened surface containing embedded metal particles. These particles act as nuclei for the crystal growth during a subsequent plating process (e.g., copper plating, gold plating, nickel plating, silver plating, zinc plating, tin plating, etc.). The laser activatable additive generally includes oxide crystals, which may include two or more metal oxide cluster configurations within a definable crystal formation. For example, the overall crystal formation may have the following general formula:

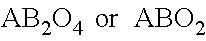

wherein,
A is a metal cation having a valance of 2 or more, such as cadmium, chromium, manganese, nickel, zinc, copper, cobalt, iron, magnesium, tin, titanium, etc., as well as combinations thereof; and
B is a metal cation having a valance of 3 or more, such as antimony, chromium, iron, aluminum, nickel, manganese, tin, etc., as well as combinations thereof.

Typically, A in the formula above provides the primary cation component of a first metal oxide cluster and B provides the primary cation component of a second metal oxide cluster. These oxide clusters may have the same or different structures. In one embodiment, for example, the first metal oxide cluster has a tetrahedral structure and the second metal oxide cluster has an octahedral cluster. Regardless, the clusters may together provide a singular identifiable crystal type structure having heightened susceptibility to electromagnetic radiation. Examples of suitable oxide crystals include, for instance, MgAl₂O₄, ZnAl₂O₄, FeAl₂O₄, CuFe₂O₄, CuCr₂O₄, MnFe₂O₄, NiFe₂O₄, TiFe₂O₄, FeCr₂O₄, MgCr₂O₄, tin/antimony oxides (e.g., (Sb/Sn)O₂), and combinations thereof. Copper chromium oxide (CuCr₂O₄) is particularly suitable for use in the present invention and is available from Shepherd Color Co. under the designation “Shepherd Black 1GM.” In some cases, the laser activatable additive may also have a core-shell configuration, such as described in WO 2018/130972. In such additives, the shell component of the additive is typically laser activatable, while the core may be any general compound, such as an inorganic compound (e.g., titanium dioxide, mica, talc, etc.).

When employed, laser activatable additives typically constitute from about 0.1 wt. % to about 30 wt. %, in some embodiments from about 0.5 wt. % to about 20 wt. %, and in some embodiments, from about 1 wt. % to about 10 wt. % of the polymer composition. Of course, the polymer composition may also be free (i.e., 0 wt. %) of such laser activatable additives, such as spinel crystals, or such additives may be present in only a small concentration, such as in an amount of about 1 wt. % or less, in some embodiments about 0.5 wt. % or less, and in some embodiments, from about 0.001 wt. % to about 0.2 wt. %.

### E. Other Optional Additives

A wide variety of other additional additives can also be included in the polymer composition, such as lubricants, thermally conductive fillers, pigments (e.g., carbon black), antioxidants, stabilizers, surfactants, waxes, flame retardants, anti-drip additives, nucleating agents (e.g., boron nitride), tribological agents (e.g., fluoropolymers), antistatic fillers (e.g., carbon nanotubes, carbon fibers, ionic liquids, carbon black, etc.), dielectric fillers, flow modifiers (e.g., aluminum trihydrate), and other materials added to enhance properties and processability. Lubricants, for example, may be employed in the polymer composition that are capable of withstanding the processing conditions of the liquid crystalline polymer without substantial decomposition. Examples of such lubricants include fatty acids esters, the salts thereof, esters, fatty acid amides, organic phosphate esters, and hydrocarbon waxes of the type commonly used as lubricants in the processing of engineering plastic materials, including mixtures thereof. Suitable fatty acids typically have a backbone carbon chain of from about 12 to about 60 carbon atoms, such as myristic acid, palmitic acid, stearic acid, arachic acid, montanic acid, octadecinic acid, parinric acid, and so forth. Suitable esters include fatty acid esters, fatty alcohol esters, wax esters, glycerol esters, glycol esters and complex esters. Fatty acid amides include fatty primary amides, fatty secondary amides, methylene and ethylene bisamides and alkanolamides such as, for example, palmitic acid amide, stearic acid amide, oleic acid amide, N,N′-ethylenebisstearamide and so forth. Also suitable are the metal salts of fatty acids such as calcium stearate, zinc stearate, magnesium stearate, and so forth, hydrocarbon waxes, including paraffin waxes, polyolefin and oxidized polyolefin waxes, and microcrystalline waxes. Particularly suitable lubricants are acids, salts, or amides of stearic acid, such as pentaerythritol tetrastearate, calcium stearate, or N,N′-ethylenebisstearamide. When employed, the lubricant(s) typically constitute from about 0.05 wt. % to about 1.5 wt. %, and in some embodiments, from about 0.1 wt. % to about 0.5 wt. % (by weight) of the polymer composition.

The components used to form the polymer composition may be combined together using any of a variety of different techniques as is known in the art. In one particular embodiment, for example, the bio-LCP and other optional additives may be melt processed as a mixture within an extruder to form the polymer composition. The mixture may be melt-kneaded in a single-screw or multi-screw extruder at a temperature of from about 250° C. to about 450° C. In one embodiment, the mixture may be melt processed in an extruder that includes multiple temperature zones. The temperature of individual zones is typically set within about −60° C. to about 25° C. relative to the melting temperature of the liquid crystalline polymer. By way of example, the mixture may be melt processed using a twin screw extruder such as a Leistritz 18-mm co-rotating fully intermeshing twin screw extruder. A general purpose screw design can be used to melt process the mixture. In one embodiment, the mixture including all of the components may be fed to the feed throat in the first barrel by means of a volumetric feeder. In another embodiment, different components may be added at different addition points in the extruder, as is known. For example, the liquid crystalline polymer may be applied at the feed throat, and certain additives (e.g., dielectric filler) may be supplied at the same or different temperature zone located downstream therefrom. Regardless, the resulting mixture can be melted and mixed then extruded through a die. The extruded polymer composition can then be quenched in a water bath to solidify and granulated in a pelletizer followed by drying.

Despite containing bio-LCP, the polymer composition may nevertheless exhibit a variety of properties that are similar to those formed with conventional types of liquid crystalline polymers. For example, the polymer composition may exhibit excellent melt processability. For example, the polymer composition may have an ultralow melt viscosity, such as from about 0.1 to about 100 Pa-s, in some embodiments from about 0.2 to about 75 Pa-s, in some embodiments from about 0.5 to about 65 Pa-s, in some embodiments from about 0.1 to about 50 Pa-s, in some embodiments from about 0.2 to about 45 Pa-s, in some embodiments from about 0.5 to about 40 Pa-s, and in some embodiments, from about 1 to about 35 Pa-s, determined at a shear rate of 1,000 seconds⁻¹ and temperature of about 15° C. greater than the melting temperature of the polymer composition in accordance with ISO 11443:2021. The polymer composition may also have excellent thermal properties. The melting temperature of the composition may, for instance, be from about 280° C. to about 400° C., in some embodiments from about 290° C. to about 380° C., and in some embodiments, from about 300° C. to about 350° C. Even at such melting temperatures, the ratio of the deflection temperature under load (“DTUL”), a measure of short term heat resistance, to the melting temperature may still remain relatively high. For example, the ratio may range from about 0.5 to about 1.00, in some embodiments from about 0.6 to about 0.95, and in some embodiments, from about 0.65 to about 0.85. The specific DTUL values may, for instance, be about 200° C. or more, in some embodiments about 220° C. or more, in some embodiments from about 230° C. to about 300° C., and in some embodiments, from about 240° C. to about 280° C. Such high DTUL values can, among other things, allow the use of high speed and reliable surface mounting processes for mating the structure with other components.

The polymer composition may also possess a high impact strength, which is useful when forming thin layers. The composition may, for instance, possess a Charpy notched impact strength of about 0.5 kJ/m² or more, in some embodiments from about 1 to about 60 kJ/m², in some embodiments from about 2 to about 50 kJ/m², and in some embodiments, from about 5 to about 45 kJ/m², as determined at a temperature of 23° C. in accordance with ISO Test No. ISO 179-1:2010. The tensile and flexural mechanical properties of the composition may also be good. For example, the polymer composition may exhibit a tensile strength of from about 20 to about 500 MPa, in some embodiments from about 50 to about 400 MPa, and in some embodiments, from about 70 to about 350 MPa; a tensile break strain of about 0.4% or more, in some embodiments from about 0.5% to about 10%, and in some embodiments, from about 0.6% to about 3.5%; and/or a tensile modulus of from about 5,000 MPa o about 20,000 MPa, in some embodiments from about 8,000 MPa to about 20,000 MPa, and in some embodiments, from about 10,000 MPa to about 20,000 MPa. The tensile properties may be determined at a temperature of 23° C. in accordance with ISO Test No. 527:2019. The polymer composition may also exhibit a flexural strength of from about 20 to about 500 MPa, in some embodiments from about 50 to about 400 MPa, and in some embodiments, from about 100 to about 350 MPa; a flexural elongation of about 0.4% or more, in some embodiments from about 0.5% to about 10%, and in some embodiments, from about 0.6% to about 3.5%; and/or a flexural modulus of from about 5,000 MPa o about 20,000 MPa, in some embodiments from about 8,000 MPa to about 20,000 MPa, and in some embodiments, from about 10,000 MPa to about 15,000 MPa. The flexural properties may be determined at a temperature of 23° C. in accordance with 178:2019.

Once formed, the polymer composition may be molded into any of a variety of different shaped parts using techniques as is known in the art. For example, the shaped parts may be molded using a one-component injection molding process in which dried and preheated plastic granules are injected into the mold. Regardless of the molding technique employed, the polymer composition is well-suited for forming electronic parts having a small dimensional tolerance. Such parts, for example, generally contain at least one micro-sized dimension (e.g., thickness, width, height, etc.), such as from about 500 micrometers or less, in some embodiments from about 50 to about 450 micrometers, and in some embodiments, from about 100 to about 400 micrometers.

One such part is a fine pitch electrical connector. More particularly, such electrical connectors are often employed to detachably mount a central processing unit (“CPU”) to a printed circuit board. The connector may contain insertion passageways that are configured to receive contact pins. These passageways are defined by opposing walls, which may be formed from a thermoplastic resin. To help accomplish the desired electrical performance, the pitch of these pins is generally small to accommodate a large number of contact pins required within a given space. This, in turn, requires that the pitch of the pin insertion passageways and the width of opposing walls that partition those passageways are also small. For example, the walls may have a width of from about 500 micrometers or less, in some embodiments from about 50 to about 450 micrometers, and in some embodiments, from about 100 to about 400 micrometers. The polymer composition of the present invention is particularly well suited to form the walls of a fine pitch connector. In addition to or in lieu of the walls, it should also be understood that any other portion of a housing of the connector may also be formed from the polymer composition. For example, the connector may also include a shield that encloses the housing. Some or all of the shield may be formed from the polymer composition. For example, the housing and the shield can each be a one-piece structure unitarily molded from the polymer composition. Likewise, the shield can be a two-piece structure that includes a first shell and a second shell, each of which may be formed from the polymer composition.

Of course, the polymer composition may also be used in a wide variety of other components. For example, the polymer composition may be molded into a planar substrate for use in an electronic component. The substrate may be thin, such as having a thickness of about 500 micrometers or less, in some embodiments from about 50 to about 450 micrometers, and in some embodiments, from about 100 to about 400 micrometers. In one embodiment, for example, the planar substrate may be applied with one or more conductive elements using a variety of known techniques (e.g., laser direct structuring, electroplating, etc.). The conductive elements may serve a variety of different purposes. In one embodiment, for example, the conductive elements form an integrated circuit, such as those used in SIM cards. In another embodiment, the conductive elements form antennas of a variety of different types, such as antennae with resonating elements that are formed from patch antenna structures, inverted-F antenna structures, closed and open slot antenna structures, loop antenna structures, monopoles, dipoles, planar inverted-F antenna structures, hybrids of these designs, etc. The resulting antenna structures may be incorporated into the housing of a relatively compact portable electronic component, such as described above, in which the available interior space is relatively small.

A planar substrate that is formed form the polymer composition described above may also be employed in other applications. For example, in one embodiment, the planar substrate may be used to form a base of a compact camera module (“CCM”), which is commonly employed in wireless communication devices (e.g., cellular phone). The compact camera module may contain a lens assembly that overlies a base. The base, in turn, overlies an optional main board. Due to their relatively thin nature, the base and/or main board are particularly suited to be formed from the polymer composition as described above. The lens assembly may have any of a variety of configurations as is known in the art, and may include fixed focus-type lenses and/or auto focus-type lenses. In one embodiment, for example, the lens assembly is in the form of a hollow barrel that houses lenses, which are in communication with an image sensor positioned on the main board and controlled by a circuit. The barrel may have any of a variety of shapes, such as rectangular, cylindrical, etc. In certain embodiments, the barrel may also be formed from the polymer composition and have a wall thickness within the ranges noted above. It should be understood that other parts of the camera module may also be formed from the polymer composition. For example, a polymer film (e.g., polyester film) and/or thermal insulating cap may cover the lens assembly. In some embodiments, the film and/or cap may also be formed from the polymer composition.

Yet other possible electronic components that may employ the polymer composition include, for instance, cellular telephones, laptop computers, small portable computers (e.g., ultraportable computers, netbook computers, and tablet computers), wrist-watch devices, pendant devices, headphone and earpiece devices, media players with wireless communications capabilities, handheld computers (also sometimes called personal digital assistants), remote controllers, global positioning system (GPS) devices, handheld gaming devices, battery covers, speakers, camera modules, integrated circuits (e.g., SIM cards), housings for electronic devices, electrical controls, circuit breakers, switches, power electronics, printer parts, etc.

The following test methods may be used to determined one or more of the properties identified above.

Melt Viscosity: The melt viscosity (Pa-s) may be determined in accordance with ISO 11443:2021 at a shear rate of 400 s⁻¹ or 1,000 s⁻¹ and temperature 15° C. above the melting temperature (e.g., about 325° C.) using a Dynisco LCR7001 capillary rheometer. The rheometer orifice (die) may have a diameter of 1 mm, length of 20 mm, L/D ratio of 20.1, and an entrance angle of 180°. The diameter of the barrel may be 9.55 mm+0.005 mm and the length of the rod may be 233.4 mm.

Melting Temperature: The melting temperature (“Tm”) may be determined by differential scanning calorimetry (“DSC”) as is known in the art. The melting temperature is the differential scanning calorimetry (DSC) peak melt temperature as determined by ISO 11357-3:2018. Under the DSC procedure, samples were heated and cooled at 20° C. per minute as stated in ISO Standard 10350 using DSC measurements conducted on a TA Q2000 Instrument.

Deflection Temperature Under Load (“DTUL”): The deflection under load temperature may be determined in accordance with ISO 75-2:2013 (technically equivalent to ASTM D648). More particularly, a test strip sample having a length of 80 mm, thickness of 10 mm, and width of 4 mm may be subjected to an edgewise three-point bending test in which the specified load (maximum outer fibers stress) was 1.8 Megapascals. The specimen may be lowered into a silicone oil bath where the temperature is raised at 2° C. per minute until it deflects 0.25 mm (0.32 mm for ISO Test No. 75-2:2013).

Tensile Modulus, Tensile Stress, and Tensile Elongation: Tensile properties may be tested according to ISO 527:2019 (technically equivalent to ASTM D638). Modulus and strength measurements may be made on the same test strip sample having a length of 80 mm, thickness of 10 mm, and width of 4 mm. The testing temperature may be 23° C., and the testing speeds may be 1 or 5 mm/min.

Flexural Modulus, Flexural Stress, and Flexural Elongation: Flexural properties may be tested according to ISO 178:2019 (technically equivalent to ASTM D790). This test may be performed on a 64 mm support span. Tests may be run on the center portions of uncut ISO 3167 multi-purpose bars. The testing temperature may be 23° C. and the testing speed may be 2 mm/min.

Charpy Impact Strength: Charpy properties may be tested according to ISO 179-1:2010 (technically equivalent to ASTM D256-10, Method B). This test may be run using a Type 1 specimen size (length of 80 mm, width of 10 mm, and thickness of 4 mm). When testing the notched impact strength, the notch may be a Type A notch (0.25 mm base radius). Specimens may be cut from the center of a multi-purpose bar using a single tooth milling machine. The testing temperature may be 23° C.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A method for forming a bio-dihydroxyphenol monomer for use in a liquid crystalline polymer, the method comprising:
providing a bio-dialkylated benzene that is derived from bio-naphtha;
oxidizing the bio-dialkylated benzene to form a bio-dialkylated benzene peroxide radical; and
subjecting the bio-dialkylated benzene peroxide radical to acid decomposition to form the bio-dihydroxyphenol.

2. The method of claim 1, wherein the bio-dialkylated benzene includes 1,4-diisopropylbenzene, 1,3-diisopropylbenzene, or a combination thereof.

3. The method of claim 1, wherein the bio-dialkylated benzene peroxide radical includes 1,4-diisopropylbenzene monohydroxyperoxide, 1,4-diisopropylbenzene dihydroxyperoxide, 1,3-diisopropylbenzene monohydroxyperoxide, 1,3-diisopropylbenzene dihydroxyperoxide, or a combination thereof.

4. The method of claim 1, wherein the acid decomposition is conducted in the presence of an acid catalyst.

5. The method of claim 1, wherein the acid decomposition is conducted in the presence of an organic solvent.

6. The method of claim 1, wherein the bio-dialkylated benzene is formed by alkylating bio-benzene in the presence of propylene, wherein the bio-benzene is derived from the bio-naphtha.

7. The method of claim 6, wherein the bio-naphtha is subjected to a steam cracking process to form the bio-benzene.

8. The method of claim 1, wherein the bio-naphtha is formed from a bio-distillate feedstock that includes a complex mixture of naturally occurring fats and/or oils.

9. The method of claim 8, wherein the bio-distillate feedstock includes a fat and/or oil derived from cotton, coconut, corn, palm, peanut, linseed, rice, rapeseed, olive, soybean, sunflower, linola, tallow, tall, castor, butter, milk, or a combination thereof.

10. The method of claim 9, wherein the bio-naphtha is formed by a method that includes fractionating the bio-distillate feedstock into a substantially liquid triglyceride phase L and a saturated or substantially saturated, solid or substantially solid triglyceride phase S, wherein the bio-naphtha is derived from the phase S.

11. The method of claim 1, wherein the bio-dihydroxyphenol monomer is bio-hydroquinone.

12-13. (canceled)

14. A bio-liquid crystalline polymer comprising repeating units derived from one or more dihydroxyphenols, optional repeating units derived from one or more aromatic hydroxycarboxylic acids, optional repeating units derived from one or more dicarboxylic acids, and optional repeating units derived from one or more biphenols, wherein the one or more dihydroxyphenols include a bio-hydroquinone derived from bio-naphtha.

15. The bio-liquid crystalline polymer of claim 14, wherein the bio-liquid crystalline polymer contains repeating units derived from bio-hydroquinone in an amount from 6 mol. % to about 40 mol. %.

16. The bio-liquid crystalline polymer of claim 14, wherein the polymer further comprises repeating units derived from 4-hydroxybenzoic acid, 2-hydroxy-6-naphthoic acid, terephthalic acid, isophthalic acid, 2,6-naphthylenedicarboxylic acid, 4,4′-biphenol, 4-aminophenol, acetaminophen, or a combination thereof.

17. The bio-liquid crystalline polymer of claim 14, wherein the thermotropic liquid crystalline polymer is wholly aromatic.

18. The bio-liquid crystalline polymer of claim 14, wherein the bio-liquid crystalline polymer has a melting temperature of from about 280° C. to about 400° C.

19. The bio-liquid crystalline polymer of claim 14, wherein the polymer has a bio-content of from about 5 wt. % to 70 wt. % based on the total weight of monomers employed in the polymer.

20. A polymer composition of claim 14, wherein the composition contains the bio-liquid crystalline polymer and one or more optional additives.

21. The polymer composition of claim 20, wherein the polymer composition has a sustainable content of from about 10 wt. % to 90 wt. % based on the total weight of the composition.
